(19) United States
(12) Patent Application Publication
(10) Pub. No.: US 2026/0191698 A1
(43) Pub. Date: Jul. 9, 2026

(54) ATTACHMENT FOR AN ABSORBENT ARTICLE

(71) Applicant: Raja Singh Tuli, Montreal (CA)

(72) Inventor: Raja Singh Tuli, Montreal (CA)

(21) Appl. No.: 19/440,756

(22) Filed: Jan. 6, 2026

Related U.S. Application Data
(60) Provisional application No. 63/743,030, filed on Jan. 8, 2025.

(51) Int. Cl. A61F 13/42; A61F 13/496

(52) U.S. Cl. CPC A61F 13/42; A61F 13/496; A61F 2013/424

(57) ABSTRACT

A detecting device configured for moisture detection and/or estimation in an absorbent article is provided that comprises a contact element configured to pierce the outer side of an absorbent portion of the absorbent article, thereby biting or penetrating into the absorbent portion and electrically coupling to the electrodes in the absorbent portion to enable the moisture detection and/or estimation in the absorbent article.

### CROSS-REFERENCE TO RELATED APPLICATIONS

This application claims priority to U.S. provisional Ser. No. 63/743,030, entitled “ATTACHMENT FOR AN ABSORBENT ARTICLE,” filed on Jan. 8, 2025. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

### Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present disclosure relates to a detecting device suitable for and/or configured to be attached to an absorbent article, especially a pull-up type absorbent article, e.g. intended for monitoring wetness in the absorbent article such as the pull-up type absorbent article.

### Description of the Related Art

Absorbent article e.g. disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, a disposable absorbent article is provided with an absorbent core capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

For many years, a variety of designs have been developed for detecting and signalling the presence of urine in an absorbent article such as a diaper. Some designs use conductive lines as sensors/electrodes to detect changes in electrical property of an absorbent article such as diaper due to urination. Such sensors/electrodes can be placed over/in/on a layer e.g. an inner layer of an absorbent article such as diaper.

### SUMMARY OF THE INVENTION

Many care facilities have no efficient way to determine, monitor, and schedule service and visits based on the real time needs of the patient. Patients are often left in their own urine and feces for extended periods of time, which may cause health problems. This leads to an increased demand for alternative incontinence management solutions.

Embodiments are presented herein of, inter alia, a wetness/saturation detection device for an absorbent article.

In an aspect of the present disclosure, a detecting device configured for moisture detection and/or estimation in an absorbent article is provided, which comprises a contact element configured to pierce the outer side of an absorbent portion of the absorbent article, thereby biting or penetrating into the absorbent portion and electrically coupling to the electrodes in the absorbent portion to enable the moisture detection and/or estimation in the absorbent article.

According to an embodiment, the contact element may consist primarily of a pin body or tongue body, with a small hook provided at its free end. Preferably, the contact element may be normally in its expanded state in which the small hook is away from the pin body or tongue body, and transition, under a pushing force, into its collapsed state in which the small hook becomes closer to the pin body or tongue body.

According to another embodiment, the contact element may be configured as clamp or clip that consists of two halves, each of which consists of a clamping body and a clamping tip, and the contact element may be normally in an open position in which the two clamping tips stay away from each other and transitions, under a force applied to the clamping body, to a close position in which the two clamping tips clamp together.

According to an embodiment, the contact element may be configured as a pin whose free end is configured to penetrate through the absorbent portion from its outer side and electrical couple an electrode in the absorbent portion, and a button/cap may be configured to be mounted to the free end of the pin from the inner side of the absorbent article.

According to an embodiment, the contact element may be configured as spring clamps that is composed of two haves that are connected together by a spring pivot, wherein each of the halves is divided by the spring pivot into a handle portion and a gripper portion, and wherein the two gripper portions are normally in a closed state because of the restoring force applied by the spring pivot, and transition into an open state under a pressing force applied onto the two handle portions that counteracts the restoring force.

According to an embodiment, the contact element may be further configured to secure the detecting device to the absorbent article in position at its absorbent portion.

According to an embodiment, the detecting device may further comprise a housing within which the electronics used for moisture detection and/or estimation are placed, and wherein at its base end, the contact element is attached to the housing and electrically coupled to the electronics, and wherein the contact element is configured to electrically couple the electronics to the electrodes in the absorbent article.

According to an embodiment, the contact element may be configured to be normally received in a cavity of the housing, and to protrude out of the cavity under an applied force in order to bite or penetrate into the absorbent portion of the absorbent article with its free end.

According to another aspect of the present disclosure, a detecting device configured for moisture detection and/or estimation in an absorbent article is provided, which comprises: a housing in which electronic components are placed and on a surface of which contact(s) is provided; and a snap lid pivotably connected to the housing at its pivoting edge and configured to cover and protect the contact(s) and to secure a section of the absorbent article once it is inserted between the housing and the snap lid from the front edge of the snap lid. The front edge is opposite to the pivoting edge. And a clasp is provided on an edge other than the pivoting and front edges of the snap lid, and a locking groove is provided on a corresponding side wall of the housing. The snap lid is configured to be securely closed by clasping the clasp to the locking groove.

This summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The various preferred embodiments of the present invention described herein can be better understood by those skilled in the art when the following detailed description is read with reference to the accompanying drawings. The components in the figures are not necessarily drawn to scale and any reference numeral identifying an element in one drawing will represent the same element throughout the drawings. The figures of the drawing are briefly described as follows.

FIG. 1A illustrates an exemplary ordinary absorbent article in an exploded perspective view;

FIG. 1B portrays the exemplary ordinary absorbent article of FIG. 1A when wearing on its wearer;

FIG. 2A illustrates a perspective view of an exemplary pull-up type absorbent article;

FIG. 2B illustrates an expanded plan view of the exemplary pull-up type absorbent article as illustrated in FIG. 2A;

FIG. 3 illustrates an exemplary contact element of a detecting device in the form of pin with hook, according to an embodiment of the present disclosure;

FIG. 4 illustrates an exemplary contact element of a detecting device in the form of clamp, according to an embodiment of the present disclosure;

FIG. 5 illustrates a detecting device according to an embodiment of the present disclosure;

FIGS. 6A and 6B illustrate another exemplary configuration of a contact element according to an embodiment of the present disclosure;

FIG. 7 illustrates an exemplary detecting device with pins that is secured to an absorbent article e.g. with aid of a button/cap, according to an embodiment of the present disclosure;

FIG. 8 illustrates an exemplary contact element of a detecting device in the form of spring clamps, according to an embodiment of the present disclosure;

FIG. 9A illustrates a perspective view of a detecting device with front clasp when its lid is opened, while FIG. 9B illustrates a plan view of the detecting device as illustrated in FIG. 9A when its lid is closed, according to an embodiment of the present disclosure;

FIGS. 10A and 10B illustrate an example detecting device with clasps provided on its side edges (also referred to as lateral edges in this disclosure), according to an embodiment of the present disclosure;

FIG. 10C is a schematic cross-sectional view of the housing 1020 as illustrated in FIG. 10A taken along C-C in FIG. 10A, while FIG. 10D is a schematic cross-sectional view of the example detecting device 1000 as illustrated in FIG. 10B taken along D-D in FIG. 10B when its lid is closed; and

FIG. 11 schematically depicts a comparison of the detecting device with front clasp as illustrated in FIGS. 9A-9B and the detecting device with clasps provided on its side edges as illustrated in FIGS. 10A-10D.

While the features described herein are susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION OF THE PREFERRED EMBODIMENTS

Absorbent article e.g. disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, a disposable absorbent article is provided with an absorbent core capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

In order for moisture detection and estimation, in particular to detect the presence and/or amount of the bodily exudates or excretions in a disposable absorbent article, in particular in its absorbent core, a number of (e.g. at least two) spaced-apart conductive lines may be provided as electrodes on e.g. the top side (i.e. the side facing the absorbent core) of the substantially liquid impervious layer along the length of the disposable absorbent article, and a detecting device e.g. a pod or an attachment may be clipped on the disposable absorbent article e.g. at its waist portion (such as e.g. at one of its waist end edges) with the contacts in the detecting device being coupled to the electrodes in the absorbent article, thereby a drive signal can be applied to one of the electrodes from the detecting device and a sense signal can be sensed from one (e.g. another one) of the electrodes by the detecting device.

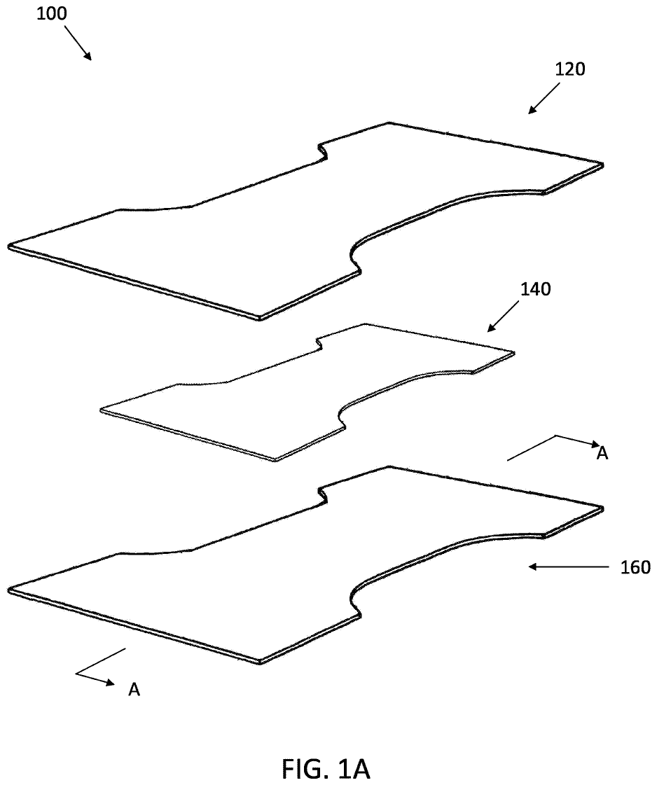

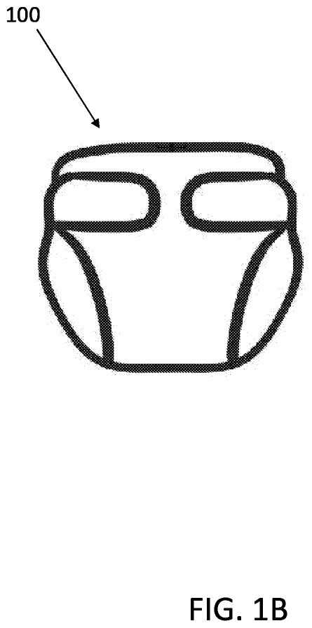

FIG. 1A illustrates a perspective view of an ordinary disposable absorbent article 100. The disposable absorbent article 100 primarily consists of an absorbent layer (e.g. pad) 140 sandwiched between two sheets/layers of nonwoven fabric, a permeable top layer/sheet 120 and an impermeable layer/sheet 160. The absorbent layer 140 is specially designed to absorb and retain body fluids, and the nonwoven fabric gives the absorbent article a comfortable shape and helps prevent leakage. Such type of ordinary absorbent articles may be made by a multi-step process in which the absorbent layer 140 is first vacuum-formed, then attached to a permeable top sheet/layer 120 and an impermeable bottom sheet/layer 160. Other layers of cloth and plastic type material may be also added in-between the absorbent layer and the two layers of non-woven fabric as intermediate layers (not shown). Various types of sensors like moisture sensors e.g. spaced-apart conductive lines, temperature sensors or other type of sensors may be provided on any of the layers (e.g. the absorbent layer, the non-woven fabric layers, or intermediate layers) of the disposable absorbent article. All of these components/layers are sealed together by application of heat or ultrasonic vibrations. Then additional attachments e.g. elastic fibers or tapes or similar attaching means like Velcro may attached to the assembled/sealed layers/sheets and act as closures to gather the edges of the absorbent article 100 into the proper shape so it fits snugly around a user's legs and crotch. When properly fitted, the absorbent article 100 will retain body fluids which pass through the permeable top sheet 120, are absorbed into the absorbent pad 140 and kept inside by the impermeable bottom sheet 160.

FIG. 1B shows the end result of the absorbent article of FIG. 1A. As an example, the layers of the absorbent article, e.g. the absorbent layer 140, the top layer/sheet 120, and the bottom layer/sheet 160 are separate components and are individually fed into an absrobent article making machine in the form of long rolls, then are joined together in the absrobent article making machine and finally cut into individual absorbent articles. First, the absorbent layer 140 is vacuum-formed, then connected to the top 120 and bottom 140 nonwoven fabric sheets. This may be accomplished e.g. by feeding the absorbent layer/pad onto a conveyor with the bottom sheet. The top sheet is then fed into place, and the compiled sheets are joined by gluing, heating, or ultrasonic welding. Finally, elastic fibers are attached to the sheets to hold the edges of the diaper and allow it to retain its shape. The long rolls are then cut into individual absorbent articles 100, folded, and packaged for shipping. The absorbent article 100 is properly fitted on a wearer; it will allow the bodily fluid to pass through the permeable top sheet and be absorbed into it.

As mentioned above, in order for moisture detection and estimation in a disposable absorbent article, a number of (e.g. at least two) spaced-apart conductive lines may be provided as electrodes on a layer (e.g. on the substantially liquid impervious layer such as on its top side (i.e. the side facing the absorbent core)) along the length of the disposable absorbent article. In the manufacturing procedure, the layer (e.g. the substantially liquid impervious layer) on which the moisture sensor e.g. the spaced-apart conductive lines is provided is fed into the absorbent article making machine in the form of long roll and joined together with other layers and then is provided with attaching means and is cut into individual absorbent article. That is, in such an absorbent article, the spaced-apart conductive lines may extend throughout the length of the absorbent article. In use, a wearer puts on such absorbent article and tapes it with the attaching means, in which the waist portion of such absorbent article is formed by the two end portions of the joined layers with aid of the attaching means, and thus the spaced-apart conductive lines may extend to the waist portion of such absorbent article as finished. Therefore, a detecting device e.g. a pod or an attachment may be clipped on such disposable absorbent article e.g. at its waist portion (such as e.g. at one of its waist end edges) with the contacts in the detecting device being coupled to the electrodes in the absorbent article, thereby a drive signal can be applied to one of the electrodes from the detecting device and a sense signal can be sensed from one (e.g. another one) of the electrodes by the detecting device.

As a specific type of absorbent article, a pull-up type absorbent article primarily consists of a waist portion such as an elasticated waist portion (e.g. front and back waist portions), and an absorbent portion. In the manufacturing procedure of such pull-up type absorbent article, its waist portion and its absorbent portion are manufacturing separately and then joined together.

Similar to other absorbent article, a pull-up type absorbent article is provided with an absorbent core capable of receiving and retaining bodily exudates or excretions, and a substantially liquid impervious layer. However, in a pull-up type absorbent article, the absorbent core and the substantially liquid impervious layer and the various sensors e.g. moisture sensors are provided only in its absorbent portion. Also similar to other disposable absorbent article, the absorbent portion of a pull-up type absorbent article consists of a layered construction, which allows the bodily exudates or excretions to be distributed and transferred to the absorbent core where they are to be retained in.

It is understood that, in a pull-up type absorbent article its waist portion (e.g. the front and back waist portions) is used to secure the pull-up type absorbent article in position about the waist of the wearer. As a result, no absorbent core is provided in the waist portion (e.g. front and back waist portions) of a pull-up type absorbent article, and thus its waist portion (e.g. front and back waist portions) is not necessarily provided with a substantially liquid impervious layer, a liquid pervious layer, and the various sensors e.g. moisture sensors such as the spaced-apart conductive lines that are provided in the absorbent portion. Instead, its waist portion (e.g. front and back waist portions) is made of a material that provides a resilient, comfortably close fit around the waist of the wearer.

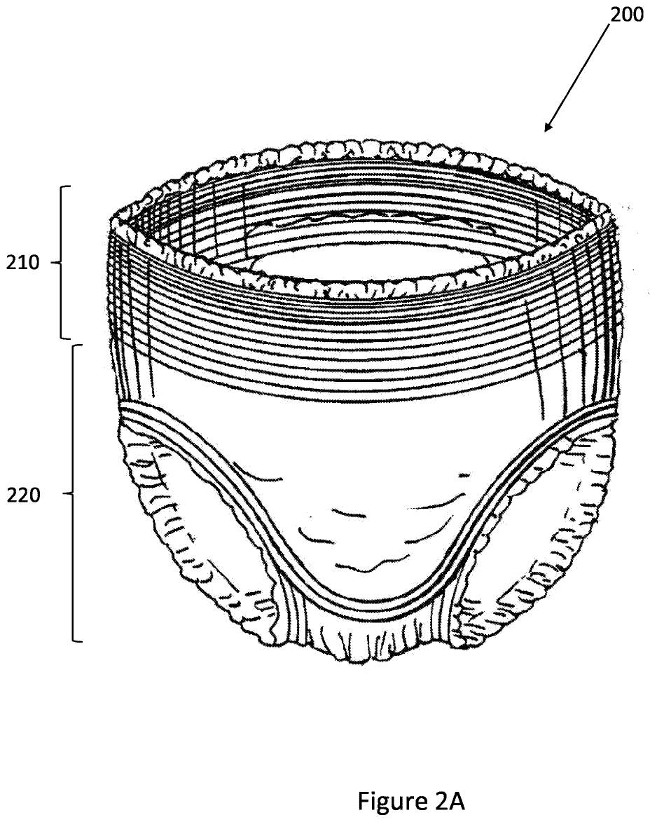

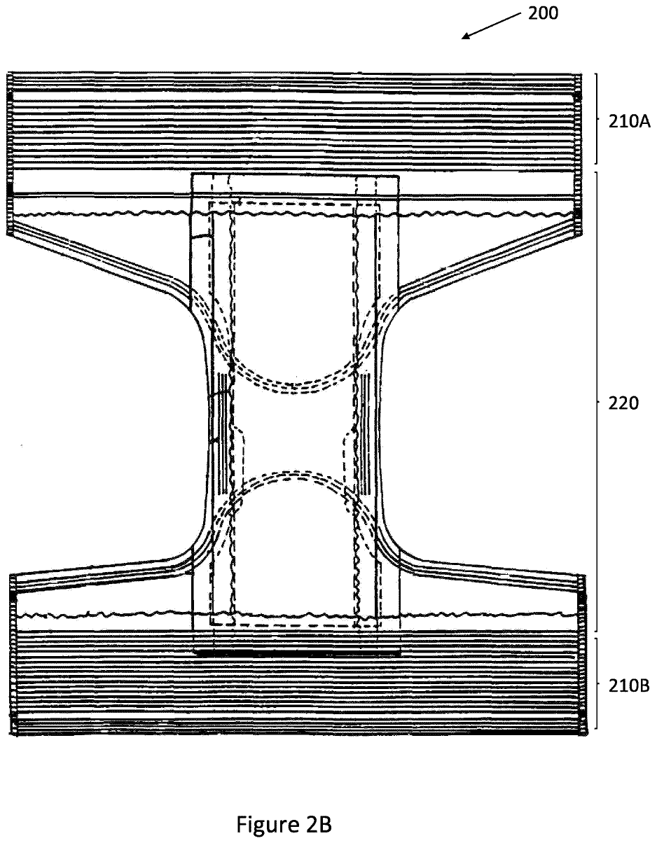

FIG. 2A illustrates a perspective view of a pull-up type absorbent article 200, while FIG. 2B illustrates an expanded plan view of the pull-up type absorbent article 200 as illustrated in FIG. 2A, according to an embodiment of the present disclosure.

As illustrated in both FIGS. 2A and 2B, a pull-up type absorbent article 200 consists primarily of a waist portion 210 and an absorbent portion 220. In the expanded plan view of FIG. 2B, the waist portion 210 is illustrated as a front waist portion 210A and a back waist portion 210B.

Since in a pull-up type absorbent article (e.g. 200, as illustrated in FIGS. 2A and 2B) the waist portions 210 (front and back waist portions 210A and 210B) and the absorbent portion 220 are manufactured separately and are made of different materials, the spaced-apart conductive lines that function as electrodes to monitor wetness/moisture in the absorbent portion will not extend to the waist portions 210. Consequently, it is impossible to detect the presence and/or amount of moisture in a pull-up type absorbent article e.g. 200, in particular in its absorbent portion e.g. 220, by attaching (e.g. clipping) a detecting device (e.g. a pod or an attachment) on its waist portion e.g. 210A or 210B.

In order to addresses this and other prior art shortcoming, the present disclosure proposes a detecting device suitable for and/or configured to be attached to an absorbent article, especially a pull-up type absorbent article, intended for monitoring wetness/moisture in the absorbent article e.g. the pull-up type absorbent article.

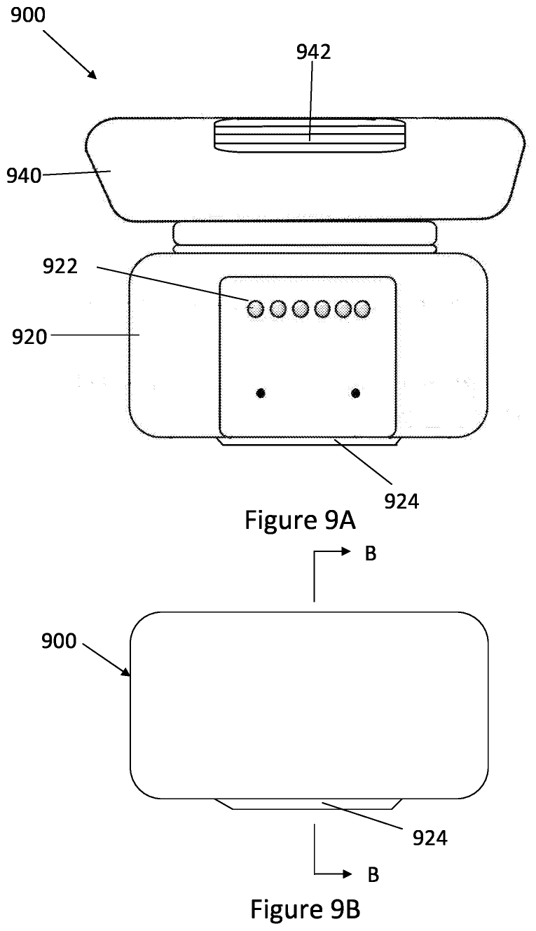

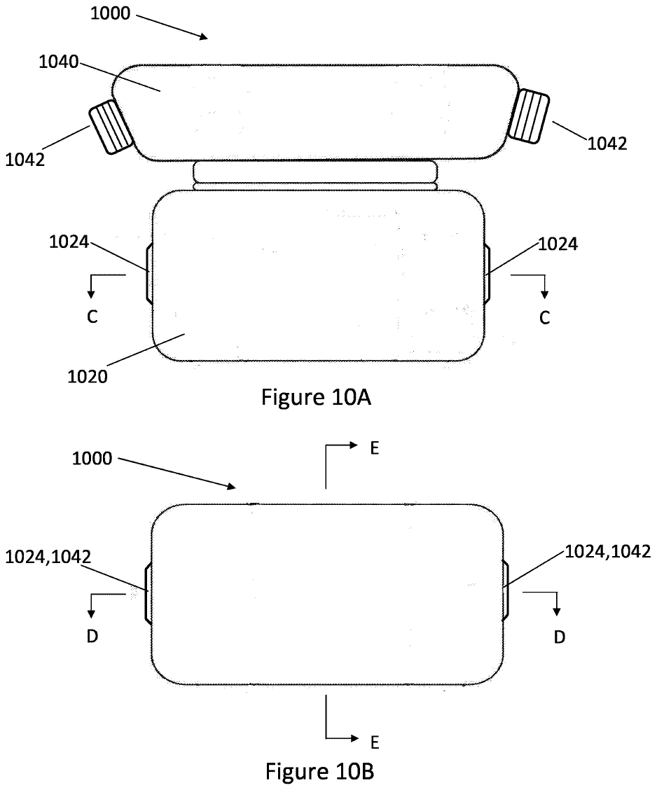

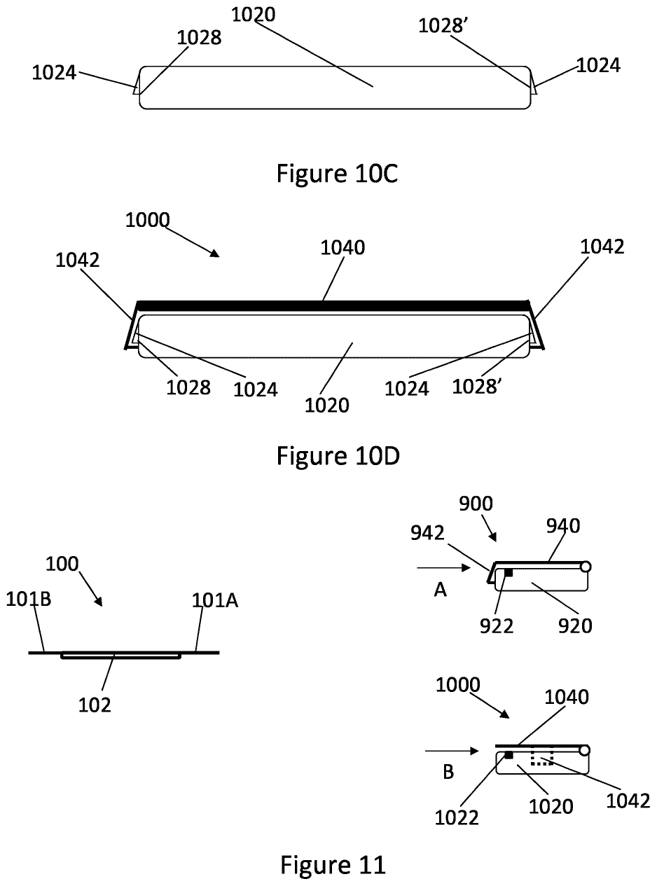

Similar to the detecting device (e.g. that as illustrated in FIGS. 9A-9B and 10A-10D) that is configured to be clipped on an ordinary absorbent article e.g. that as illustrated in FIGS. 1A and 1B at its waist portion (e.g. at one of its waist end edges), a detecting device according to an embodiment of the present disclosure comprises a housing, preferably made of plastic, within which the electronics (not shown) used for moisture detection, e.g. printed circuit board are placed. As explained above, the detecting device according to an embodiment of the present disclosure is configured to be attached to an absorbent article e.g. a pull-up type absorbent article at its absorbent portion, instead of being clipped on its waist portion (e.g. at one of its waist end edge). To this end, the detecting device according to an embodiment of the present disclosure comprises attachment element(s) that is configured to secure the detecting device on an absorbent article e.g. a pull-up type absorbent article in position at its absorbent portion. It is understood that the attachment element(s) may be any type/form of attachment element(s) that is suitable to secure the detecting device on an absorbent article at its absorbent portion.

In order for moisture detection, a detecting device according to an embodiment of the present disclosure comprises contact element(s) that is configured to pierce the outer side of the absorbent portion of an absorbent article e.g. a pull-up type absorbent article, and thus bite/penetrate into the absorbent portion and to electrically couple the electronics in the housing to the electrodes in the absorbent portion. It is understood that in addition to be used to electrically couple to the electrodes in the absorbent article (in particular, in its absorbent portion), thereby enabling the detection of moisture in the absorbent article e.g. by applying a drive signal to one of the electrodes from the electronics e.g. PCB in the housing and sensing a sense signal from one (e.g. another one) of the electrodes, the contact element(s) may also function as attachment element(s) to secure the detecting device on the absorbent article in position.

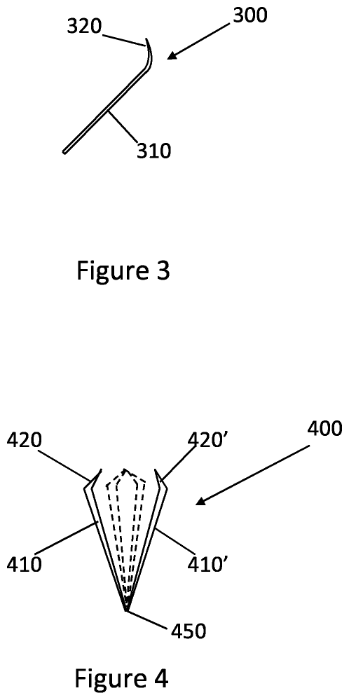

In an embodiment of the present disclosure, a contact element is configured as pin or tongue with hook at its free end. FIG. 3 illustrates an exemplary contact element of a detecting device in the form of pin with hook, according to an embodiment of the present disclosure. As illustrated in FIG. 3, a contact element 300 of a detecting device suitable for e.g. a pull-up type absorbent article is configured as a pin or tongue, according to an embodiment of the present disclosure. The contact element 300 consists primarily of a pin body or tongue body 310. At its free end, the contact element 300 is provided with a small hook 320. The other end of the contact element 300 is configured to be attached to the housing of a detecting device and to be electrically coupled to the electronics that are placed in the housing.

In use, the contact element 300 as illustrated in FIG. 3 bites (e.g. penetrates) with its free end into the absorbent portion of an absorbent article e.g. a pull-up type absorbent article, then the hook 320 of the contact element 300 remains and gets hooked in the absorbent portion, and electrically couples an electrode in the absorbent article, thereby enabling the moisture detection. It is to be noted that, since its hook 320 gets hooked in the absorbent portion of the absorbent article, the contact element 300 can secure the detecting device in position on the absorbent portion or at least facilitate such securing.

As an example, the contact element 300 may be configured to be normally received in a cavity of a detecting device's housing. In order to bite into the absorbent portion of an absorbent article, the contact element 300 is made (e.g. with aide of a push button) to protrude out of the cavity. Next the contact element 300, when protruding out of the cavity, is made to bite (e.g. penetrate) into the absorbent portion of the absorbent article with its hook 320. Then the contact element 300 retreats back into the cavity (e.g. because the pushing force applied on the push button is withdrawn) and its hook 320 remains and gets hooked in the absorbent portion of the absorbent article and electrically coupled to an electrode in the absorbent portion, thereby enabling the moisture detection in the absorbent portion of the absorbent article and/or securing the detecting device in position on the absorbent portion of the absorbent article.

As mentioned above, the contact element 300 functions as contacts to electrically couple the electronics provided within the detecting device to an electrode in the absorbent portion of an absorbent article e.g. a pull-up type absorbent article. To this end, the contact element 300 (at least its hook 320) is made of conductive material e.g. metal. In use, when being hooked in the absorbent portion of an absorbent article, the hook 320 is coupled to an electrode in the absorbent portion of the absorbent article.

In another embodiment of the present disclosure, the contact element may be configured as clamp or clip. FIG. 4 illustrates an exemplary contact element 400 of a detecting device in the form of clamp, according to an embodiment of the present disclosure. As illustrated in FIG. 4, the contact element 400 consists of two halves, each of which consists of a clamping body 410 or 410′ and a clamping tip 420 or 420′. In FIG. 4, the open position of the contact element 400 is shown in solid lines, while the close position of the contact element 400 is shown in dotted lines.

Similar to the contact element 300 in the form of pin or tongue with hook as described above with reference to FIG. 3, the base end 450 of the contact element 400 as illustrated in FIG. 4 is configured to be attached to the housing of a detecting device and to be electrically coupled to the electronics that are placed in the housing.

In use, the contact element 400 as illustrated in FIG. 4 bites (e.g. penetrates) in its open position with its clamping tips 420 and 420′ into the absorbent portion of an absorbent article e.g. a pull-up type absorbent article, and then transitions to its close position in which the clamping tips 420 and 420′ clamp together in the absorbent portion and remain in the absorbent portion and electrically couple to an electrode in the absorbent portion, thereby enabling the moisture detection in the absorbent portion and/or securing the detecting device in position on the absorbent portion.

As an example, the contact element 400 may be configured to be normally received in a cavity of a detecting device's housing, which cavity forces the contact element 400 to be in its close position. In order to bite into the absorbent portion of an absorbent article, the contact element 400 is made (e.g. with aide of a push button) to protrude out of the cavity and thus become in its open position. The contact element 400, when protruding out of the cavity and being in its open position, is made to bite (e.g. penetrate) into the absorbent portion of the absorbent article. Then the contact element 400 retreats back into the cavity and resumes its close position in the cavity with its clamping tips 420 and 420′ clamping together and remaining in the absorbent portion of the absorbent article and electrically coupling an electrode in the absorbent portion, thereby enabling the moisture detection in the absorbent portion of the absorbent article and/or securing the detecting device in position on the absorbent portion of the absorbent article.

As mentioned above, the contact element 400 functions as contacts to electrically couple the electronics placed in the detecting device to an electrode in the absorbent portion of an absorbent article e.g. a pull-up type absorbent article. To this end, the contact element 400 (at least one of its halves) is made of conductive material e.g. metal. In use, when clamping together and remaining in the absorbent portion of an absorbent article, the clamping tips 420 and 420′ are coupled to an electrode in the absorbent portion of the absorbent article.

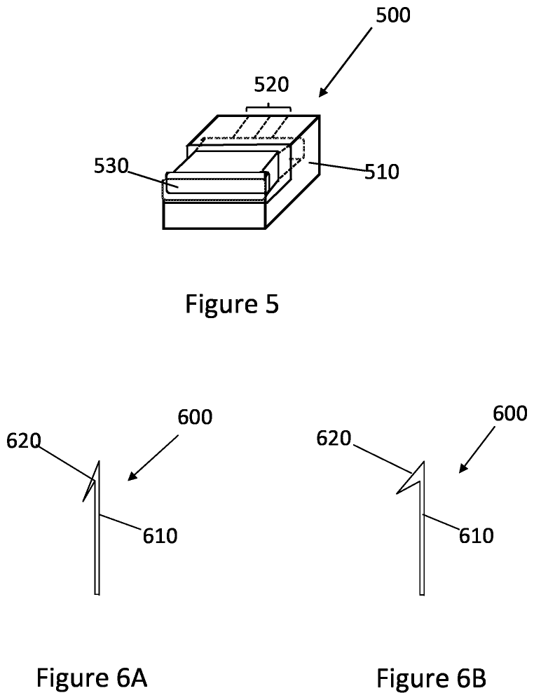

As mentioned above, the contact elements e.g. as illustrated in FIGS. 3 and/or 4 may be normally received in the housing of the detecting device. As an example, FIG. 5 illustrates a detecting device 500 suitable for e.g. a pull-up type absorbent article according to an embodiment of the present disclosure. As illustrated in FIG. 5, a detecting device 500 according to an embodiment of the present disclosure consists primarily of a housing 510. The detecting device 500 as illustrated in FIG. 5 further comprises a number of contact elements 520 that are normally received in the housing 510. Further, a push button 530 is provided on the detecting device 500 (in particular on its housing 510) that is configured to push the contact elements 520 out of the detecting device 500 (in particular out of its housing 510) in an embodiment of the present disclosure.

In use, the contact elements 520 (e.g. 300 and/or 400 as illustrated in FIGS. 3 and/or 4) are pushed out of the housing 510 with aid of a push button 530, and bite (e.g. penetrate) into the absorbent portion of the absorbent article. Then with release of the push button 530, the contact elements 520 retreat back into the housing 510 of the detecting device, while their free ends e.g. hook 320 and/or clamping tips 420 and 420′ remain (e.g. get hooked or clamp together) in the absorbent portion and electrically couple an electrode in the absorbent portion, thereby enabling the moisture detection in the absorbent portion of the absorbent article and/or securing the detecting device 500 in position on the absorbent article e.g. the pull-up type absorbent article.

That is, the detecting device 500 can be secured in position on an absorbent article e.g. a pull-up type absorbent article, and the free ends (e.g. the hook 320 and/or the clamping tips 420 and 420′) of the contact elements 520 that remain in the absorbent portion of the absorbent article are coupled to the electrodes in the absorbent portion, which in turn enable the detection of the moisture in the absorbent portion. In particular, the electronics placed within the housing 510 of the detecting device 500 send out a drive signal that is applied to an electrode in the absorbent portion of the absorbent article via a contact element 520 of the detecting device 500, and sense a sense signal back from an (e.g. another) electrode in the absorbent portion of the absorbent article via a (e.g. another) contact element 520 of the detecting device 500.

In an embodiment of the present disclosure, a compartment is provided in the detecting device 500 (e.g. in its housing) within which the contact elements are received. Alternatively, a number of holes or cavities are provided in the detecting device 500 (e.g. in its housing) in which the contact elements are received respectively, in an embodiment of the present disclosure.

FIGS. 6A and 6B illustrate another exemplary configuration of a contact element according to an embodiment of the present disclosure. As illustrated in FIGS. 6A and 6B, a contact element 600 is configured also as a pin or tongue with hook at its free end, similarly with that in FIG. 3. The contact element 600 consists primarily of a pin body or tongue body 610. At its free end, the contact element 600 is provided with a small hook 620. The other end of the contact element 600 is configured to be attached to the housing of a detecting device and to be electrically coupled to the electronics that are placed in the housing. The contact element 600 is configured to be normally in its expanded state as illustrated in FIG. 6B, in which the small hook 620 is somewhat away from the pin body 620. When the contact element 600 is pushed in order to pierce the absorbent portion of an absorbent article and thus bit/penetrate into the absorbent portion, the pushing force transitions the contact element 600 into its collapsed state as illustrated in FIG. 6A, in which the small hook 620 becomes closer to the pin body 620. It is understood that the collapsed state of the contact element 600 facilitate it piercing the absorbent portion of an absorbent article and thus biting/penetrating into the absorbent portion. When the pushing force is released after the contact element 600 bits into the absorbent portion of the absorbent article, the contact element 600 transitions back to its expanded state as illustrated in FIG. 6B. It is understood that the expanded state of the contact element facilitate it getting hooked in the absorbent portion. And in order to remove a contact element 600 from the absorbent portion of an absorbent article, a pushing force is again applied to the contact element 600 which forces the contact element 600 into its collapsed state and then the contact element 600 can be easily pulled out from the absorbent portion.

The contact element 600 may be configured to be normally received in the housing of the detecting device and protrude out from the housing e.g. under a pushing force when the detecting device is to be attached to and/or detached from an absorbent article, similarly as the contact elements as illustrated in FIGS. 3, 4, and 5. Also, the contact element 600 may be configured to be protruding from the housing of the detecting device.

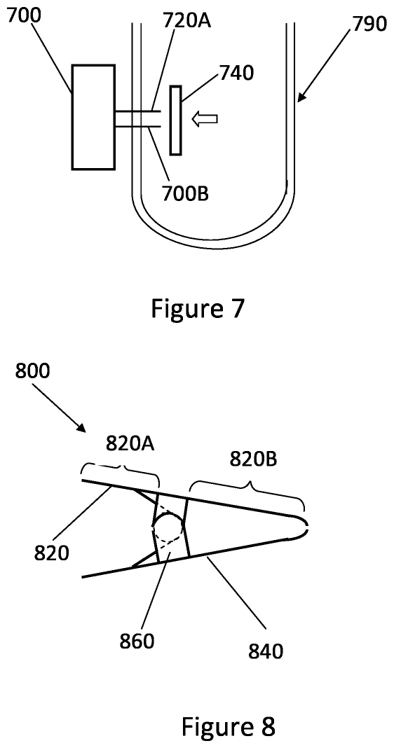

In another embodiment of the present disclosure, a contact element of a detecting device is configured as a pin, which is configured to penetrate through the absorbent portion of an absorbent article from its outer side (i.e. the side opposite to the wearer of the absorbent article) and electrical couple an electrode in the absorbent portion, and a button/cap is configured to be mounted on the pin from the inner side (i.e. the side facing the wearer) of the absorbent article. FIG. 7 illustrates an exemplary detecting device 700 with pins 720A and 720B that is secured to an absorbent article 790 e.g. with aid of a button/cap 740 at its absorbent portion. As illustrated in FIG. 7, a detecting device 700 is provided with pins e.g. two pins 720A and 720B that penetrate the absorbent portion of an absorbent article 790 from its outer side and electrically couple the respective electrodes in the absorbent portion. A cap/button 740 is configured to be mounted on the pins from the inner side of the absorbent article in order to avoiding the pins contacting/piercing the wearer's skin and/or secure the detecting device in position. The pins 720A and 720B may be configured to be normally received in the housing of the detecting device 700 and protrude out from the housing e.g. under a pushing force when the detecting device 700 is to be attached to an absorbent article 790, similarly as the contact elements as illustrated in FIGS. 3, 4, 5, and 6. Also, the pins 720A and 720B may be configured to be protruding from the housing of the detecting device 700.

In a further embodiment of the present disclosure, a contact element is configured as spring clamps, as illustrated in FIG. 8. As illustrated in FIG. 8, a contact element 800 is composed of two haves 820 and 840 that are connected together by a spring pivot 860. The half 820 is divided by the spring pivot 860 into a handle portion 820A and a gripper portion 820B, as illustrated in FIG. 8. Similarly, the half 840 is divided by the spring pivot 860 into a handle portion and a gripper portion. And the spring pivot 860 applies a restoring force on the two halves 820 and 840 to keep the two gripper portions in a closed state. In use, a pressing force is applied onto the two handle portions e.g. 820A which counteracts the restoring force applied by the spring pivot 860 and forces the two gripper portions e.g. 820B into an open state. When in the open state, the tips of the two gripper portions e.g. 820B is pushed to pierce the outer side of the absorbent portion of an absorbent article and thus penetrate into the absorbent portion, then the pressing force is released, consequently the tips of the two gripper portions return to the closed state and electrically couple an electrode in the absorbent article. It is understood that in addition to electrically coupling an electrode in the absorbent article (in particular, in its absorbent portion), the closed state of the two gripper portions of the contact element 800 may also secure the detecting device on the absorbent article in position or at least facilitate such securing.

And in order to remove a contact element 800 from the absorbent portion of an absorbent article, a pressing force is again applied to the handle portions, which forces the two gripper portions into the open state and then the contact element 800 can be easily pulled out from the absorbent portion.

As mentioned above, the contact element 800 functions as contacts to electrically couple the electronics placed within the detecting device to an electrode in the absorbent portion of an absorbent article e.g. a pull-up type absorbent article. To this end, the contact element 800 (at least one of its two halves e.g. 820 and 840) is made of conductive material e.g. metal. In use, when gripping in the absorbent portion of an absorbent article, the tip(s) is coupled to an electrode in the absorbent portion of the absorbent article.

As mentioned above, a detecting device is configured to cooperate with electrodes provided in an absorbent article, for moisture detection in the absorbent article. In another aspect of the present disclosure, a detecting device is configured as an enclosure with a snap lid. FIG. 9A illustrates a perspective view of a detecting device 900 when its lid is opened, while FIG. 9B illustrates a plan view of the detecting device 900 when its lid is closed, according to an embodiment of the present disclosure.

Referring to FIG. 9A now, a detecting device 900 has an insulating housing 920, preferably made of plastic, in which electronic components e.g. a printed circuit board (PCB) and optionally a battery are placed. The housing 920 may be in the form of a cuboid, with two surfaces (upper surface and bottom surface) of largest surface area facing opposite each other and four side walls connecting the two surfaces. There provides on the upper surface of the housing 920 a number of contact terminals 922, e.g. the equal number of contact terminals 922 as the electrodes provided in the absorbent article. The detecting device 900 is adapted to apply a drive signal to one of the electrodes from the electronics e.g. PCB in the housing and sensing a sense signal from one (e.g. another one) of the electrodes, thereby enabling the moisture detection in the absorbent article. In an embodiment of the present disclosure and as illustrated in FIG. 9A, the detecting device 900 is provided with a snap lid 940 to cover and protect the contact terminals 922 and to secure a section of the absorbent article with electrodes once it is inserted inside the detecting device 900, in particular between the snap lid 940 and the housing 920, thereby securing/clipping the detecting device on the absorbent article e.g. at its waist portion. As illustrated in FIG. 9A, the snap lid 940, at its pivoting side/edge (one of its (e.g. long) sides/edges, and in this description also referred to as back edge), may be connected to the housing 920 by pivotable joint at a pivoting (e.g. a long) side of the upper surface of the housing. The snap lid 940 is securely closed by further clasping or locking a clasp or tab 942 into a locking groove 924. As an example and as illustrated in FIGS. 9A and 9B, a clasp or tab 942 may be provided on the front side (i.e. the side/edge opposite to the pivoting side/edge, e.g. the other long side/edge, and in this disclosure also referred to as front edge) of the snap lid 940 from where a section of an absorbent article is inserted/slid into the detecting device 900, in particular between its snap lid 940 and its housing 920, and correspondingly a locking groove 924 may be provided on the corresponding front side wall (i.e. the side wall opposite to that along the pivoting side of the upper surface) of the housing 920.

It is understood that when a section of an absorbent article is inserted/slid into a detecting device e.g. 900, in particular between its snap lid e.g. 940 and its housing e.g. 920, a front clasp or tap e.g. 942 provided on the front edge of the snap lid will take up additional section of the absorbent article in order to clasp or be locked to the corresponding locking groove. Further it is understood that it is difficult or even impossible for the section with absorbent material of an absorbent article to be insert or slid into a detecting device or be clasped by a clasp or tab, due to its thickness. Therefore, in order to be used with a detecting device with a front clasp or tab provided on the front edge, an absorbent article needs to be provided with a large distance between the waist end edge of the absorbent article and the outermost edge of its absorbent material, i.e. a wide edge/waist portion without absorbent material.

However, not all absorbent articles are provided with a wide edge/waist portion without absorbent material. In order to be used with an absorbent article with a narrow edge/waist portion without absorbent material and/or in order to reduce the width of the section of an absorbent article that is necessary for clasping by a detecting device, clasp(s) or tab(s) may be provided on the side(s)/edge(s) other than the front and back/pivoting edges (e.g. one or both of the short sides/edges, and in this disclosure, referred to as lateral edges or side edges) of the detecting device's snap lid, and correspondingly, locking groove(s) may be provided on the corresponding lateral side wall(s) (i.e. the side wall(s) other than those along the front and back/pivoting sides of the upper surface) of the detecting device's housing, in another embodiment of the present disclosure.

According to an embodiment of the present disclosure, FIGS. 10A and 10B illustrate an example detecting device 1000 that has an insulating housing 1020, preferably made of plastic, in which electronic components e.g. a printed circuit board (PCB) and optionally a battery are placed. Similarly as that as illustrated in FIGS. 9A and 9B, the housing 1020 may be in the form of a cuboid, with two surfaces (upper surface and bottom surface) of largest surface area facing opposite each other and four side walls connecting the two surfaces. Also, the detecting device 1000 is provided with a snap lid 1040 to cover and protect the contact terminals and to secure a section of the absorbent article with electrodes once it is inserted inside the detecting device 1000, thereby securing/clipping the detecting device on the absorbent article e.g. at its waist portion. The snap lid 1040 is securely closed by further clasping or locking the clasps or tabs 1042 to the locking grooves 1024. In the embodiment as illustrated in FIGS. 10A and 10B, two clasps or tabs 1042 are provided on the lateral edges of the snap lid 1040, rather than on its front edge from where a section of an absorbent article is inserted/slid into the detecting device 1000, in particular between its snap lid 1040 and its housing 1020, and correspondingly two locking grooves 1024 are provided on the lateral side walls of the housing 1020.

FIG. 10C is a schematic cross-sectional view of the housing 1020 as illustrated in FIG. 10A taken along C-C in FIG. 10A. As illustrated in FIG. 10C, two locking grooves 1024 are provided on the two lateral side walls 1028 and 1028′. It is to be noted that, the locking grooves 1024 are illustrated as protrusions on the lateral side walls, though it is possible for them to take a different form e.g. a form of recess. FIG. 10D is a schematic cross-sectional view of the example detecting device 1000 as illustrated in FIG. 10B taken along D-D in FIG. 10B, when its lid is closed. In FIG. 10D, the cross-sectional view of the housing 1020 is similar to that as illustrated in FIG. 10C. As the snap lid 1040 is closed, it is illustrated as being on top of the housing 1020, with its two claps/tabs 1042 that are provided on its lateral edges being clasped or locked to the locking grooves 1024. It is understood that a section of the absorbent article with electrodes is inserted into the space between the snap lid 1040 and the housing 1020.

FIG. 11 schematically depicts a comparison of the detecting device 900 with front clasp as illustrated in FIGS. 9A-9B and the detecting device 1000 with clasps provided on its lateral edges as illustrated in FIGS. 10A-10B. The left of FIG. 11 schematically depicts a schematic cross-sectional view of an absorbent article 100 as illustrated in FIG. 1A taken along A-A in FIG. 1A, after the various layers 120, 140, and 160 are assembled. As illustrated, the waist portion 101 (in particular, the front waist portion 101A and the back waist portion 101B) without absorbent material e.g. 140 is thin, while the absorbent portion 102 is thicker because of the presence of absorbent material/layer e.g. 140.

The upper right of FIG. 11 schematically depicts a schematic cross-sectional view of the detecting device 900 as illustrated in FIGS. 9A-9B taken along B-B in FIG. 9B, while the lower right of FIG. 11 schematically depicts a schematic cross-sectional view of the detecting device 1000 as illustrated in FIGS. 10A-10D taken along E-E in FIG. 10B. In both the cross-sectional views of the detecting device 900 and 1000 as illustrated in the right of FIG. 11, the pivotable joint is illustrated as a circle at the top-right corner of the cross-sectional view of the respective housing 920 and 1020, which pivotably connects the snap lid 940/1040 to the housing 920/1020.

As explained above, a waist portion (e.g. the front waist portion 101A) of an absorbent article 100 is inserted between the snap lid 940/1040 and the housing 920/1020 from the front edge (i.e. the edge opposite to the pivotable joint) of the detecting device 900/1000 (in FIG. 11, in the direction of the arrow A or B), in order for the conductive lines provided on/in the waist portion to contact/couple the contact terminals 922/1022 provided on the upper surface (i.e. the surface facing the snap lid) of the housing 920/1020.

As described above and as illustrated in FIG. 11, for the detecting device 900, a front clasp or tab 942 is provided on the front edge of the snap lid 940 and clasps or is locked into a locking groove 924 provided on the front side wall of the housing 920 after a waist portion e.g. 101A of the absorbent article 100 is inserted/slid between the snap lid 940 and the housing 920 from the front edge of the detecting device 900. As mentioned above, the front clasp or tap e.g. 942 will take up additional section of the waist portion when clasping or being locked to the corresponding locking groove, which makes it difficult or even impossible for the waist portion (in particular, the conductive lines provided thereon/therein) to couple/contact the contact terminals 922 on the upper surface of the housing 920 because the absorbent portion 102 prevents the absorbent article 100 (in particular, its waist portion) from being inserted further due to its thickness.

On the other hand, for detecting device 1000, clasp(s) or tab(s) 1042 is provided on the lateral edge(s) of the snap lid 940 and clasps or is locked into locking groove(s) 1024 provided on the lateral side wall(s) of the housing 1020 after a waist portion e.g. 101A of the absorbent article 100 is inserted/slid between the snap lid 940 and the housing 920 from the front edge of the detecting device 1000. It is to be noted that in FIG. 11, clasp(s) or tab(s) 1042 is illustrated with dotted lines because it is not visible in the cross-sectional view. It is understood that the clasp(s) or tap(s) e.g. 1042 will not impact or prevent the absorbent article 100 (in particular, its waist portion 101) from being inserted further, because the absorbent article 100 is inserted from the front edge of the detecting device 1000 while the clasp(s)/tab(s) 1042 and the locking groove(s) 1024 are provided on the lateral edge(s) of the detecting device 1000. Therefore, when comparing with the detecting device 900, the detecting device 1000 facilitates the insertion of the absorbent article (in particular, its waist portion) into the detecting device (i.e. between its snap lid and its housing) and the coupling/contact of the conductive lines on/in the absorbent article with the contact terminals of the detecting device.

Although the embodiments above have been described in considerable detail, numerous variations and modifications will become apparent to those skilled in the art once the above disclosure is fully appreciated. It is intended that the following claims be interpreted to embrace all such variations and modifications.

What is claimed is:

1. A detecting device configured for moisture detection and/or estimation in an absorbent article, comprising a contact element configured to pierce the outer side of an absorbent portion of the absorbent article, thereby biting or penetrating into the absorbent portion and electrically coupling to the electrodes in the absorbent portion to enable the moisture detection and/or estimation in the absorbent article.

2. The detecting device according to claim 1, wherein the contact element consists primarily of a pin body or tongue body, with a small hook provided at its free end.

3. The detecting device according to claim 2, wherein the contact element is normally in its expanded state in which the small hook is away from the pin body or tongue body, and transitions, under a pushing force, into its collapsed state in which the small hook becomes closer to the pin body or tongue body.

4. The detecting device according to claim 1, wherein the contact element is configured as clamp or clip that consists of two halves, each of which consists of a clamping body and a clamping tip, and wherein the contact element is normally in an open position in which the two clamping tips stay away from each other and transitions, under a force applied to the clamping body, to a close position in which the two clamping tips clamp together.

5. The detecting device according to claim 1, wherein the contact element is configured as a pin whose free end is configured to penetrate through the absorbent portion from its outer side and electrical couple an electrode in the absorbent portion, and wherein a button/cap is configured to be mounted to the free end of the pin from the inner side of the absorbent article.

6. The detecting device according to claim 1, wherein the contact element is configured as spring clamps that is composed of two haves that are connected together by a spring pivot, wherein each of the halves is divided by the spring pivot into a handle portion and a gripper portion, and wherein the two gripper portions are normally in a closed state because of the restoring force applied by the spring pivot, and transition into an open state under a pressing force applied onto the two handle portions that counteracts the restoring force.

7. The detecting device according to claim 1, wherein the contact element is further configured to secure the detecting device to the absorbent article in position at its absorbent portion.

8. The detecting device according to claim 1, further comprising a housing within which the electronics used for moisture detection and/or estimation are placed, and wherein at its base end, the contact element is attached to the housing and electrically coupled to the electronics, and wherein the contact element is configured to electrically couple the electronics to the electrodes in the absorbent article.

9. The detecting device according to claim 8, wherein the contact element is configured to be normally received in a cavity of the housing, and to protrude out of the cavity under an applied force in order to bite or penetrate into the absorbent portion of the absorbent article with its free end.

10. A detecting device configured for moisture detection and/or estimation in an absorbent article, comprising:
a housing in which electronic components are placed and on a surface of which contact(s) is provided; and
a snap lid pivotably connected to the housing at its pivoting edge and configured to cover and protect the contact(s) and to secure a section of the absorbent article once it is inserted between the housing and the snap lid from the front edge of the snap lid, the front edge being opposite to the pivoting edge,
wherein a clasp is provided on an edge other than the pivoting and front edges of the snap lid while a locking groove is provided on a corresponding side wall of the housing, and wherein the snap lid is configured to be securely closed by clasping the clasp to the locking groove.
